(19) United States
(12) Patent Application Publication
(10) Pub. No.: US 2025/0064410 A1
(43) Pub. Date: Feb. 27, 2025

(54) MEDICAL INFORMATION PROCESSING APPARATUS, PET APPARATUS, AND MEDICAL INFORMATION PROCESSING METHOD

(71) Applicant: CANON MEDICAL SYSTEMS CORPORATION, Otawara-shi (JP)

(72) Inventors: Kentaro KAYAHARA, Nasushiobara (JP); Ryusei SAIKI, Nasushiobara (JP); Yasunobu YAMADA, Nasushiobara (JP)

(73) Assignee: CANON MEDICAL SYSTEMS CORPORATION, Otawara-shi (JP)

(21) Appl. No.: 18/801,925

(22) Filed: Aug. 13, 2024

(30) Foreign Application Priority Data
Aug. 25, 2023 (JP) 2023-137542

(51) Int. Cl. A61B 6/03; A61B 6/00; A61B 6/46; G16H 20/40

(52) U.S. Cl. CPC A61B 6/037; A61B 6/461; A61B 6/5217; G16H 20/40

(57) ABSTRACT

A medical information processing apparatus according to an embodiment includes processing circuitry. The processing circuitry obtains a morphological image representing a morphology of a subject and a PET image of the subject, and detects a region containing each of sites of the subject included in the PET image with reference to the morphological image. The processing circuitry determines whether additional imaging is to be performed for at least one of the sites included in the PET image, based on determination-criterion information containing a determination criterion, and outputs information according to a result of the determination. The determination criterion is a criterion based on which performing or not performing additional imaging is determined and is set for each of the sites.

### CROSS-REFERENCE TO RELATED APPLICATIONS

This application is based upon and claims the benefit of priority from Japanese Patent Application No. 2023-137542, filed on Aug. 25, 2023; the entire contents of which are incorporated herein by reference.

### FIELD

Embodiments described herein relate generally to a medical information processing apparatus, a PET apparatus, and a medical information processing method.

### BACKGROUND

Traditionally, healthcare professionals generally determine whether additional imaging is to be performed, with reference to a positron emission tomography (PET) image. Using a PET apparatus, it takes a certain amount of time for additional imaging of the same site of a subject. For additional imaging of a different site of the subject, the PET apparatus requires a certain amount of setting time and imaging time.

However, the PET apparatus captures the subject after a prescribed time has passed from administration of a medical agent into the subject. Healthcare professionals are, however, not allowed to take a large amount of time for additional imaging due to a strict schedule management imposed. In this regard, there is a request for development of a technique to alleviate the burden on the healthcare professionals with respect to the additional imaging.

### BRIEF DESCRIPTION OF THE DRAWINGS

FIG. 1 is a schematic block diagram illustrating an exemplary configuration of a PET-CT apparatus according to a first embodiment;

FIG. 2 is a flowchart illustrating one example of an imaging process to be performed by the PET-CT apparatus according to the first embodiment;

FIG. 3 is a schematic block diagram illustrating an exemplary configuration of a PET-CT apparatus according to a second embodiment; and

FIG. 4 is a flowchart illustrating one example of an imaging process to be performed by the PET-CT apparatus according to the second embodiment.

### DETAILED DESCRIPTION

According to an embodiment, a medical information processing apparatus includes processing circuitry. The processing circuitry obtains a morphological image representing a morphology of a subject and a PET image of the subject, and detects a region containing each of sites of the subject included in the PET image with reference to the morphological image. The processing circuitry determines whether additional imaging is to be performed for at least one of the sites included in the PET image, based on determination-criterion information containing a determination criterion, and outputs information according to a result of the determination. The determination criterion is a criterion based on which performing or not performing additional imaging is determined and is set for each of the sites.

Hereinafter, exemplary embodiments of a medical information processing apparatus, a PET apparatus, and a medical information processing method will be described with reference to the accompanying drawings. In the following embodiments, parts, portions, elements, or functions denoted by the same reference numerals are considered to perform same or similar operation, and an overlapping explanation thereof will be omitted when appropriate.

Throughout this disclosure, a medical information processing apparatus according to some embodiments is defined to be a positron emission tomography (PET)-computed tomography (CT) apparatus for the sake of specificity. A PET-CT apparatus 1 is one example of PET apparatus.

### First Embodiment

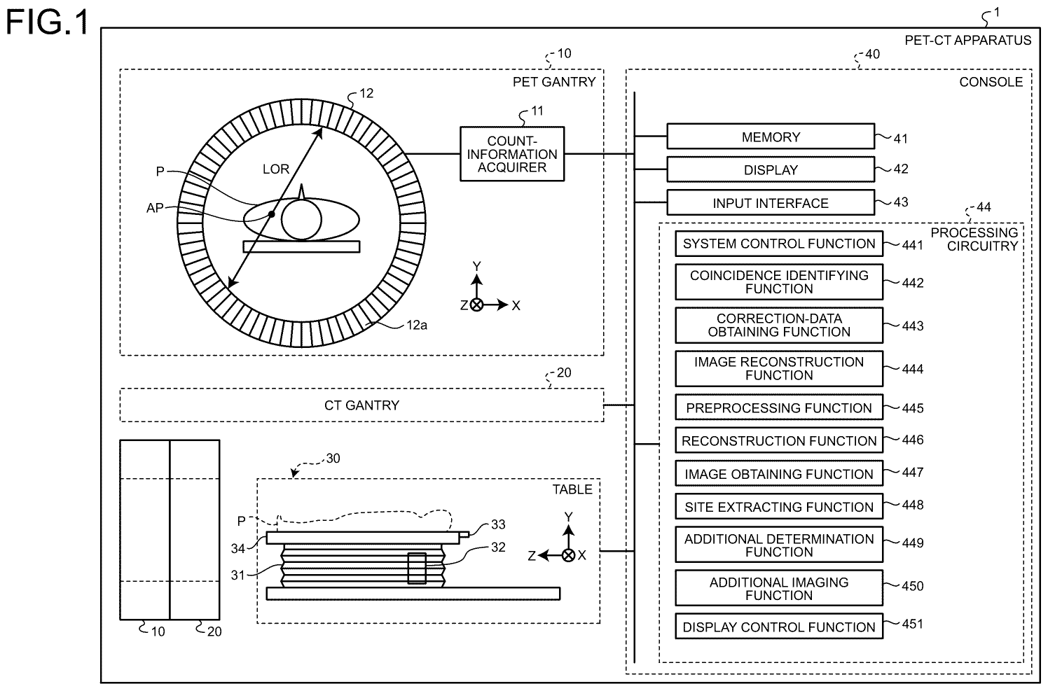

FIG. 1 illustrates an exemplary configuration of the PET-CT apparatus 1 according to a first embodiment. As illustrated in FIG. 1, the PET-CT apparatus 1 includes a PET gantry 10, a CT gantry 20, a table 30, and a console 40.

In the present embodiment, the axis of a PET detector 12 or the longitudinal direction of a table-top 33 of the table 30 is defined as a Z-axis direction. An axial direction orthogonal to the Z-axis direction and horizontal to the floor is defined as an X axis direction. An axial direction orthogonal to the Z-axis direction and vertical to the floor is defined as a Y-axis direction. Note that FIG. 1 depicts multiple PET gantries 10 and CT gantries 20 for convenience of explanation, however, the PET-CT apparatus 1 includes one PET gantry 10 and one CT gantry 20 alone in reality.

The PET gantry 10 includes a count-information acquirer 11 and the PET detector 12. The PET detector 12 detects gamma rays (pair annihilation gamma rays) emitted from inside a subject P. The PET detector 12 includes multiple PET detector modules 12a arranged in ring form around the subject P.

The PET detector modules 12a generate scintillation in accordance with the gamma rays emitted from inside the subject P, and emit light. The PET detector modules 12a detect the light and convert the light into an electric signal according to the energy.

The count-information acquirer 11 generates count information from the output signals of the PET detector modules 12a. The count-information acquirer 11 then stores the count information in the memory 41. The count-information acquirer 11 can be, for example, implemented by a processor. For example, the count-information acquirer 11 generates and acquires count information from the output signals of the PET detector modules 12a. The count information contains gamma-ray detected positions, energy values, and detection time, for example. The count-information acquirer 11 transmits, to the console 40, the count information in data format called single list mode data prior to coincidence counting.

In more detail, the count-information acquirer 11 identifies a gamma-ray emitted position (position of scintillation) in the scintillator. If scintillator pixels and optical sensors are coupled together on a one-to-one basis, the count-information acquirer 11 identifies a light-emitting position by identifying a scintillator pixel corresponding to the optical sensor having emitted a pulse. Alternatively, the count-information acquirer 11 may infer the light-emitting position by barycentric calculation of outputs from a smaller number of optical sensors, if the smaller number of optical sensors are associated with a larger number of scintillator pixels.

The count-information acquirer 11 infers the energy from the output pulses from the optical sensors. For example, the count-information acquirer 11 infers the energy by an energy integral method in which the ranges of the output pulses exceeding a predetermined threshold are integrated and translated into energy. For another example, the count-information acquirer 11 infers the energy by a time-over-threshold (ToT) method in which the temporal range where the output pulses exceed a predetermined threshold is translated into energy. The count-information acquirer 11 determines timing at which the output pulse from the optical sensor exceeds a predetermined threshold as a detection time.

The CT gantry 20 is an apparatus including an imaging system that irradiates the subject P with X-rays to acquire detection data of X-rays having transmitted the subject P. The CT gantry 20 obtains the detection data to generate correction data (e.g., attenuation map, p-map) for use in reconstruction of PET image data.

The table 30 is an apparatus on which the subject P to be scanned is laid and moved, and includes a base 31, table drive circuitry 32, the table-top 33, and a table-top support frame 34. The base 31 serves as a housing that movably supports the table-top support frame 34 in the vertical direction. The table drive circuitry 32 may be a motor or an actuator for moving the table-top 33 on which the subject P is lying along the longitudinal axis of the table-top 33. The table drive circuitry 32 moves the table-top 33 under the control of the console 40, the PET gantry 10, or the CT gantry 20. The table-top 33 located on the top of the table-top support frame 34 is a plate on which the subject P is to be laid. The table drive circuitry 32 may move the table-top support frame 34 along the longitudinal axis of the table-top 33, in addition to the table-top 33.

For scanning the subject P, the table 30 may move the table-top 33 by the step & shoot method in which scanning and table-top movement are alternated or by the continuous table motion method in which the table-top 33 is being moved while being scanned.

The console 40 includes the memory 41, a display 42, an input interface 43, and processing circuitry 44. Although the console 40 is independent from the PET gantry 10 and the CT gantry 20 herein, the console 40 or part of the elements of the console 40 may be included in the PET gantry 10 or the CT gantry 20.

The memory 41 can be implemented by, for example, a semiconductor memory device as a random access memory (PAM) or a flash memory, a hard disk, or an optical disk. The memory 41 stores therein projection data, CT image data, and PET image data, for example. For another example, the memory 41 contains computer programs for causing the circuitry of the PET-CT apparatus 1 to perform various kinds of functions. The memory 41 may be implemented by a server farm (cloud) connected to the PET-CT apparatus 1 via a network.

Further, the memory 41 stores therein determination-criterion information containing a determination criterion set for each site to determine whether additional imaging is to be performed. The determination-criterion information refers to information including a determination criterion set for each site to determine whether additional imaging is to be performed.

The site refers to a body part of the subject P. For example, the site can be a particular organ, a particular bone, a particular muscle, or a particular blood vessel, or a portion indicating a particular body tissue. In addition, the site can be a segment of an organ, such as the large bowl and the small bowl as segments of the bowls. The large bowl may also be segmented into the colon and the rectum. Moreover, the site can be a part with no specific name given. For example, the site can be a separated part such as the upstream side or downstream side of a blood vessel of a certain organ.

For example, the determination criterion for performing or not performing additional imaging includes an image pattern with need for additional imaging and an image pattern without need for additional imaging. The determination criterion also indicates whether the maximum standardized uptake value (SUV) of an associated site is a threshold or less.

In addition, it is preferable to set the details of the determination criterion in accordance with an associated site. For example, with respect to the gastrointestinal tract as an associated site, the determination criterion may be set to a relatively wide range of values for the purpose of examination of clearance of the gastrointestinal tract. With respect to the urinary bladder as an associated site, the determination criterion may be set to values indicating no determination to be made as to additional imaging since the urinary bladder is a site relatively difficult to determine a need for additional imaging. Also, with respect to the urinary bladder as an associated site, the determination criterion may be set to easily reachable values in order to determine whether an administered medical agent is sufficiently spreading inside the body.

The determination criterion is not limited to the SUV values and may be set to other kinds of information. Other examples of the determination criterion include a standard deviation or a mean value of pixel values in a site appearing on a maximum intensity projection (MIP) image, a variation in count rate of each pixel in a site during imaging, two-dimensional A or three-dimensional A, and amount of remaining noise after noise reduction.

Further, the determination criterion is not limited to the state of an associated site and may be set to states of multiple sites. In the case of cancer metastasis examination, for example, it is preferable to examine two or more sites. In such a case the determination criterion may be set to the states of two or more sites. For example, the determination criterion may be set to the states of both the liver and the gallbladder or the states of both the stomach and the lymphatic node.

The determination-criterion information may contain a degree of priority as to additional imaging for each site. There are, for example, readily diagnosable sites and diagnostically difficult sites on PET images. In view of this, the determination-criterion information may include a degree of priory set for each site in accordance with a degree of diagnostic readiness. This allows the PET-CT apparatus 1 to identify a site to be subject to additional imaging in accordance with the priority, when determining that additional imaging is to be performed if time permits. The degree of priority may be set in accordance with a degree of another condition, in addition to the degree of diagnostic readiness.

The display 42 displays various kinds of information. For example, the display 42 outputs medical images (CT images and PET images) generated by the processing circuitry 44 and a graphical user interface (GUI) that allows the operator to perform various operational inputs. Examples of the display 42 include a liquid crystal display (LCD), an organic electroluminescence display (OELD), a plasma display, or any other displays when appropriate. Alternatively, the display 42 may be included in the PET gantry 10 or the CT gantry 20. The display 42 may be a desktop type or may include a tablet terminal wirelessly communicable with the console 40.

The input interface 43 receives various operational inputs from the operator to convert the operational inputs into electrical signals and output the electrical signals to the processing circuitry 44. As an example, the input interface 43 receives, from the operator, an acquisition condition for acquiring projection data, a reconstruction condition for reconstructing CT images, an image processing condition for generation of post-processed images from CT images, and else. Examples of the input interface 43 include a mouse, a keyboard, a trackball, a switch, a button, a joystick, a touchpad, and a touch panel display, as appropriate.

In the present embodiment, the input interface 43 is not limited to the one including a physical operational component such as a mouse, a keyboard, a trackball, a switch, a button, a joystick, a touchpad, and a touch panel display. Other examples of the input interface 43 include electrical-signal processing circuitry that receives an electrical signal corresponding to an operational input from an external input device separated from the apparatus to output the electrical signal to the processing circuitry 44. The input interface 43 is one example of an input unit. The input interface 43 may be included in the PET gantry 10 or the CT gantry 20. Alternatively, the input interface 43 may include a tablet terminal wirelessly communicable with the console 40.

The processing circuitry 44 controls the PET-CT apparatus 1 as a whole. The processing circuitry 44 includes, for example, a system control function 441, a coincidence identifying function 442, a correction-data obtaining function 443, an image reconstruction function 444, a preprocessing function 445, a reconstruction function 446, an image obtaining function 447, a site extracting function 448, an additional determination function 449, an additional imaging function 450, and a display control function 451. According to one embodiment, processing and functions implemented by the system control function 441, coincidence identifying function 442, correction-data obtaining function 443, image reconstruction function 444, preprocessing function 445, reconstruction function 446, image obtaining function 447, site extracting function 448, additional determination function 449, additional imaging function 450, and display control function 451 are stored in computer-executable program format in the memory 41. The processing circuitry 44 serves as a processor that retrieves and executes the programs from the memory 41 to implement the respective functions. In other words, having retrieved the respective programs, the processing circuitry 44 includes the functions shown in the processing circuitry 44 of FIG. 1.

In FIG. 1, the single processor implements the system control function 441, coincidence identifying function 442, correction-data obtaining function 443, image reconstruction function 444, preprocessing function 445, reconstruction function 446, image obtaining function 447, site extracting function 448, additional determination function 449, additional imaging function 450, and display control function 451, by way of example. However, the processing circuitry 44 can be constituted of a combination of multiple independent processors, so that the processors can individually execute the programs to implement the respective functions. In FIG. 1, a single piece of memory circuitry such as the memory 41 stores therein computer programs corresponding to the respective functions, as an example. Alternatively, multiple pieces of memory circuitry may be disposed in a distributed manner to allow the processing circuitry 44 to individually retrieve the corresponding programs from the pieces of memory circuitry.

The term “processor” used herein signifies, for example, circuitry such as a central processing unit (CPU), a graphical processing unit (GPU), an application specific integrated circuit (ASIC), or a programmable logic device (e.g., a simple programmable logic device (SPLD), a complex programmable logic device (CPLD), and a field programmable gate array (FPGA)). The processor retrieves and executes the computer programs from the memory 41 to implement the functions. In place of being stored in the memory 41, the computer programs may be directly embedded in the circuitry of the processor. In such a case the processor retrieves and executes the computer programs from the circuitry to implement the functions.

The system control function 441 controls the respective functions of the processing circuitry 44 in response to operational inputs from the operator given via the input interface 43.

The coincidence identifying function 442 generates list mode data by allowing respective events in single list mode data generated by the count-information acquirer 11 to mutually coincide. The coincidence identifying function 442 pairs the events detected in a predetermined time window (order of ps) to identify two substantially simultaneously detected events. The coincidence identifying function 442 may be included not in the processing circuitry 44 but in the count-information acquirer 11. Thus, the count-information acquirer 11 may transmit count information in list mode data format to the console 40.

The correction-data obtaining function 443 obtains projection data from the CT gantry 20 to obtain a CT image as correction data by reconstruction of the projection data. The correction data is used in reconstruction of PET image data. Examples of the correction data include an attenuation map or a μ-map.

The image reconstruction function 444 generates PET image data by reconstructing the count information generated by the coincidence identifying function 442. As an example, the image reconstruction function 444 performs reconstruction by maximum likelihood-expectation maximization (ML-EM) or ordered subset-expectation maximization (OS-EM) being fast ML-EM.

The preprocessing function 445 subjects the detection data output from the CT gantry 20 to preprocessing including, for example, logarithm conversion, offset correction, sensitivity correction among the channels, and beam hardening correction, to generate data. Data before preprocessing (detection data) and data after preprocessing may be collectively referred to as projection data.

The reconstruction function 446 generates CT image data by performing reconstruction processing to the projection data generated by the preprocessing function 445 by filtered back projection (FBP) or iterative reconstruction, for example.

The image obtaining function 447 obtains CT image data representing the morphology of the subject P and PET image data corresponding to the CT image data. The image obtaining function 447 is one example of an obtaining unit. A CT image based on CT image data is one example of a morphological image. The PET gantry 10 and the CT gantry 20 are disposed in juxtaposition in the direction into which the subject P is inserted, i.e., the Z-axis direction. The PET gantry 10 and the CT gantry 20 capture the subject P when inserted into the bore. Through this imaging, the image obtaining function 447 obtains PET image data generated by the image reconstruction function 444 based on the output data from the PET gantry 10 as well as CT image data generated by the reconstruction function 446 based on the output data from the CT gantry 20.

The site extracting function 448 extracts each site of the subject P included in the PET image data, based on the CT image data.

Specifically, the site extracting function 448 detects the location of each site of the subject P included in the CT image data obtained by the image obtaining function 447. In other words, the site extracting function 448 detects a region containing each site of the subject P included in the PET image data, with reference to the CT image data. The site extracting function 448 is one example of a detector unit. The site extracting function 448 may detect the location of each site of the subject P from the CT image data by any method. For example, the site extracting function 448 may detect the location of each site of the subject P using a trained model, anatomical landmarks, or any other method.

Voxels of the CT image data and voxels of the PET image data of the subject P inserted in the CT gantry 20 and the PET gantry 10 are mutually associated. As such, the site extracting function 448 can determine which site of the subject P the voxels of the PET image data represent, by detecting the locations of the respective sites of the subject P appearing on the CT image data. In this manner the site extracting function 448 can extract the respective sites of the subject P included in the PET image data by detecting the locations of the respective sites of the subject P included in the CT image data.

The additional determination function 449 determines whether additional imaging is to be performed with respect to at least one of the sites included in the PET image data extracted by the site extracting function 448, based on the determination-criterion information stored in the memory 41. The additional determination function 449 is one example of a determiner unit. For example, the additional determination function 449 determines whether additional imaging is to be performed for each of the sites included in the PET image data. Thus, the additional determination function 449 determines whether or not, according to the determination-criterion information, the site concerned is defined as the one to undergo additional imaging.

Specifically, the additional determination function 449 uses, for example, a trained model for the determination. The trained model has learned by algorithms such as B-branch method, support vector machine (SVMV), deep neural network (DNN), or logistic regression, using the determination-criterion information as training data. In addition, the additional determination function 449 may make rule-based or case-based determinations.

The additional determination function 449 may apply three or more classifications, in addition to the two classifications of additional imaging to be performed and not to be performed. For example, the additional determination function 449 may determine a degree of priority as to additional imaging of a site concerned. For another example, the additional determination function 449 may apply classifications as additional imaging needed, additional imaging to be performed if a remaining time is available, and additional imaging unneeded. With respect to the site determined as additional imaging to be performed if a remaining time is available, the additional determination function 449 determines whether additional imaging is to be performed according to a time taken for additional imaging and the degree of priority set in the determination-criterion information.

Alternatively, the additional determination function 449 may determine whether additional imaging of the site is to be performed based on the degree of priority set in the determination-criterion information, a length of time defined by a medical agent administered to the subject P, and a length of time ensured for capturing a PET image of the subject P. The length of time defined by a medical agent administered to the subject P refers to a time during which PET imaging is feasible, as defined by date and time when administering the medical agent to the subject P, and a type and a dose of the medical agent administered. The length of time ensured for capturing a PET image of the subject P refers to a time allocated to the subject P concerned in the schedule of the PET-CT apparatus 1 for multiple subjects P. Thus, the additional determination function 449 determines that additional imaging is necessary for the site concerned, if PET imaging is feasible within the time defined by the medical agent administered to the subject P and within the time ensured for capturing a PET image of the subject P.

When the determination criterion set in the determination-criterion information includes the states of multiple sites, the additional determination function 449 determines whether additional imaging is to be performed for each of the sites in the PET image data according to the states of the corresponding sites and the determination criterion defining the states of multiple sites.

When the additional determination function 449 determines that additional imaging is necessary, the additional imaging function 450 allows additional imaging of the site concerned. The additional imaging function 450 is one example of an execution unit. Specifically, the additional imaging function 450 generates an examination plan for additional imaging of an imaging area including the site concerned. This additional imaging function 450 is one example of a generator unit. The examination plan refers to information representing PET imaging settings of the PET gantry 10. That is, the PET gantry 10 performs PET imaging following the examination plan.

For example, the additional imaging function 450 generates an examination plan for allowing the site to undergo additional imaging to be located at approximately the center of PET image data. The additional imaging function 450 then transmits the examination plan to the PET gantry 10. In other words, the additional imaging function 450 outputs information corresponding to a result of the determination by the additional determination function 449. This additional imaging function 450 is an exemplary output unit. In this manner the additional imaging function 450 causes the PET gantry 10 to perform imaging of the site concerned. In addition the additional imaging function 450 may also transmit the examination plan to the CT gantry 20 to cause the CT gantry 20 to perform imaging. Thus, the examination plan may also be information representing CT imaging settings of the CT gantry 20. Alternatively, the examination plan may be pieces of information separately prepared for the PET gantry 10 and for the CT gantry 20.

As for multiple sites to be subject to additional imaging, the additional imaging function 450 may generate an examination plan for an imaging area containing the multiple sites to be captured by imaging once or twice or more. For another example, numerical values indicating a degree of need for additional imaging of multiple sites may be calculated. In such a case the additional imaging function 450 may generate an examination plan for imaging the multiple sites in order indicated by the degree of need for additional imaging.

The display control function 451 displays the PET image generated by the additional imaging on the display 42. The display control function 451 also displays the PET image as a subject of the determination on additional imaging obtained by the image obtaining function 447 and the PET image generated by additional imaging in a comparable manner. The display control function 451 is an exemplary display control unit.

For example, the display control function 451 may display the PET image as a subject of the determination on additional imaging and the PET image generated by additional imaging in juxtaposition, in a superimposed manner, or in an operationally switchable manner.

Further, the display control function 451 may generate a difference image representing differences between the PET image as a subject of the determination on additional imaging and the PET image generated by additional imaging. For another example, when additional imaging has been performed three times or more, the display control function 451 may display first, second, and third PET images being a subject of the determination on additional imaging in a comparable manner.

Now, the processing to be performed by the PET-CT apparatus 1 is described.

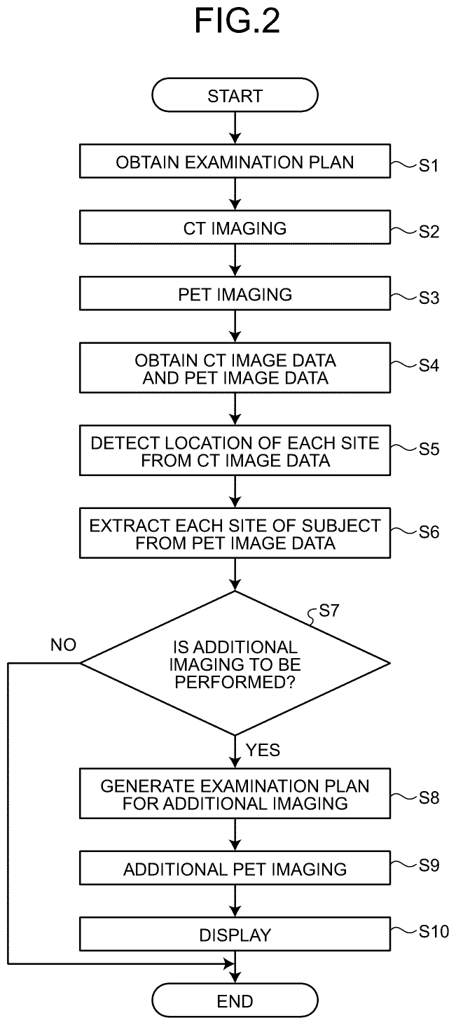

FIG. 2 is a flowchart illustrating the imaging process that the PET-CT apparatus 1 performs according to the first embodiment, as an example.

The system control function 441 obtains an examination plan as to initial imaging by each of the CT gantry 20 and the PET gantry 10 (step S1). For example, the system control function 441 may generate and obtain an examination plan in accordance with a user's operation or obtain a prepared examination plan.

The system control function 441 transmits the examination plan to the CT gantry 20 to instruct the CT gantry 20 to perform a helical scan, for instance (step S2).

The system control function 441 transmits the examination plan to the PET gantry 10 to instruct the PET gantry 10 to perform imaging (step S3).

The image obtaining function 447 obtains CT image data based on output data from the CT gantry 20 and PET image data based on output data from the PET gantry 10 (step S4).

The site extracting function 448 detects the location of each site of the subject P contained in the CT image data (step S5).

The site extracting function 448 extracts each site of the subject P from the PET image data, with reference to the detected location of each site of the subject P (step S6).

The additional determination function 449 determines for each site of the subject P whether additional imaging is to be performed based on the determination-criterion information (step S7). When determining not to perform additional imaging (No at step S7), the additional determination function 449 completes the imaging process.

When the additional determination function 449 determines to perform additional imaging (Yes at step S7), the additional imaging function 450 generates an examination plan for additional imaging of an imaging area containing the site concerned (step S8).

The additional imaging function 450 transmits the examination plan to the PET gantry 10 to cause the PET gantry 10 to perform additional PET imaging (step S9).

The display control function 451 displays the initial PET image captured at step S3 and the PET image resulting from the additional imaging in a comparable manner (step S10).

Thereby, the PET-CT apparatus 1 completes the imaging process.

As described above, the PET-CT apparatus 1 of the first embodiment obtains a CT image representing the morphology of the subject P and a PET image of the subject P. The PET-CT apparatus 1 then detects a region containing each site of the subject P in the PET image, with reference to the CT image. The PET-CT apparatus 1 determines whether additional imaging is to be performed for at least one of the sites included in the PET image, based on determination-criterion information containing a determination criterion, set for each of the sites of the subject P, based on which performing or not performing additional imaging is determined. The PET-CT apparatus 1 outputs, to the PET gantry 10, an examination plan for instructing the PET gantry 10 to perform additional imaging, according to a result of the determination. As such, the PET-CT apparatus 1 can lessen the burden of additional imaging.

### Second Embodiment

According to a second embodiment, a PET-CT apparatus 1a issues a notification of need for additional imaging, upon determining to perform additional imaging. In response to receipt of an additional imaging instruction, the PET-CT apparatus 1a performs additional imaging.

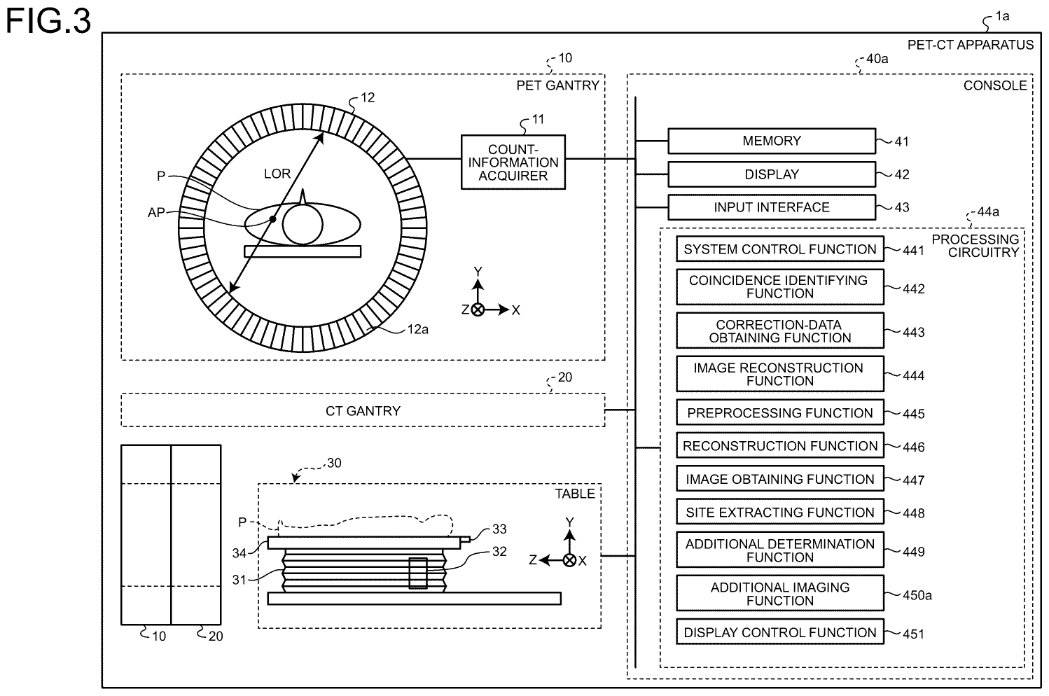

FIG. 3 illustrates an exemplary configuration of the PET-CT apparatus 1a according to the second embodiment.

Processing circuitry 44a includes an additional imaging function 450a that generates an examination plan for allowing a site to be subject to additional imaging to be located at approximately the center of PET image data, when the additional determination function 449 determines to perform additional imaging, as in the first embodiment.

The additional imaging function 450a issues a notification of need for additional imaging. The additional imaging function 450a is one example of a notifying unit. For example, the additional imaging function 450a may display a notification image that notifies the user of a need for additional imaging on the display 42. As an example, the notification image may contain a message for informing the user of a need for additional imaging. For example, the message includes a site to be subject to additional imaging, a determination criterion contained in the determination-criterion information, and a recommendation that additional imaging is to be performed in conformity with the determination criterion.

In addition, the notification image may contain the details of an examination plan created for additional imaging. In other words, the notification image can be a proposal of the created examination plan. In this regard, the system control function 441 receives an instruction for performing additional imaging under the created examination plan. The system control function 441 is one example of an operation unit. The additional imaging function 450a transmits the examination plan to the PET gantry 10 in response to receipt of the instruction for performing additional imaging under this examination plan. In this manner the additional imaging function 450a causes the PET gantry 10 to perform additional imaging based on the examination plan.

For another example, in response to a cancellation of the created examination plan, the additional imaging function 450a displays a prepared image that allows the user to manually create an examination plan. Thereby, the additional imaging function 450a can prompt the user to create an examination plan. The additional imaging function 450a may display a prepared image representing an examination plan for allowing a site being a subject of additional imaging to be located at approximately the center of the imaging area. This image display setting may be switchable by the user.

In addition, the notification image may contain a PET image obtained by previous PET imaging. The additional imaging function 450a may display the site being a subject of additional imaging in a highlighted manner on the PET image. For example, the additional imaging function 450a may add color, an image, or a message to the site being a subject of additional imaging on the PET image.

The additional imaging function 450a may notify the user via the display in a display format depending on the degree of priority of additional imaging. For instance, the additional determination function 449 may classify the sites into a site with a need for additional imaging, a site to be subject to additional imaging if time permits, and a site with no need for additional imaging. In this case, the additional imaging function 450a may display the PET image in a display format according to the resultant site classification. For example, the site with a need for additional imaging and the site to be subject to additional imaging if time permits may be displayed in red and green, respectively. The site with no need for additional imaging may be displayed without change of color. In such a case the additional imaging function 450a may highlight the site with a need for additional imaging in red and highlight the site to be subject to additional imaging if time permits in green on the PET image. The colors of the site with a need for additional imaging, the site to be subject to additional imaging if time permits, and the site with no need for additional imaging are merely exemplary. The additional imaging function 450a can highlight the PET image in any color on display. For example, the additional imaging function 450a may display the PET image in a highlighted manner such that the site with a need for additional imaging is highlighted in yellow and the site to be subject to additional imaging if time permits is highlighted in orange. Also, the additional imaging function 450a may highlight the site with no need for additional imaging in any color. The additional imaging function 450a may highlight the PET image on display in a different manner than color changing.

Now, the processing to be performed by the PET-CT apparatus 1a is explained.

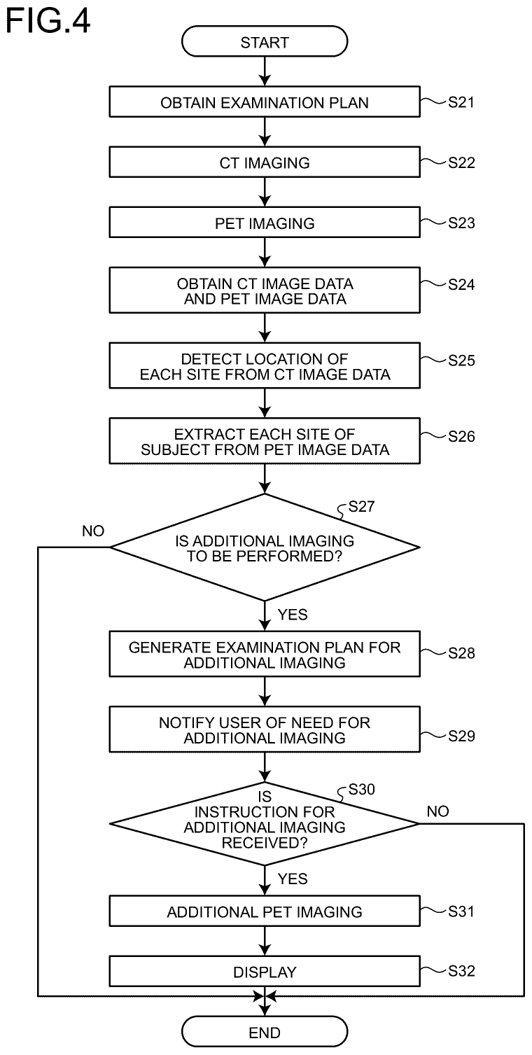

FIG. 4 is a flowchart illustrating the imaging process that the PET-CT apparatus 1a performs according to the second embodiment by way of example.

From steps S21 to S27, the processing circuitry 44a performs processing in a similar manner to that from steps S1 to S7 of the imaging process in FIG. 2.

When additional imaging is not to be performed (No at step S27), the PET-CT apparatus 1a completes the imaging process. When additional imaging is to be performed (Yes at step S27), the additional imaging function 450a generates an examination plan for additional imaging of an imaging area containing a site concerned (step S28).

The additional imaging function 450a issues a notification of a need for additional imaging (step S29).

The additional imaging function 450a determines whether to have received an instruction as to additional imaging (step S30). Upon receipt of an instruction for not performing additional imaging (No at step S30), the additional imaging function 450a completes the imaging process.

Upon receipt of an instruction for performing additional imaging (Yes at step S30), the additional imaging function 450a transmits the generated examination plan to the PET gantry 10 to cause the PET gantry 10 to perform additional PET imaging (step S31).

The display control function 451 displays the initial PET image captured at step S23 and the PET image resulting from the additional imaging in a comparable manner (step S32).

The PET-CT apparatus 1a then completes the imaging process.

As described above, the PET-CT apparatus 1a of the second embodiment issues a notification of need for additional imaging. In response to receipt of an instruction for performing additional imaging, the PET-CT apparatus 1a outputs, to the PET gantry 10, an examination plan to give an instruction for the PET gantry 10 to perform additional imaging. In this manner the PET-CT apparatus 1a can alleviate the burden of additional imaging.

### First Modification

The first embodiment and the second embodiment have described the PET-CT apparatuses 1 and 1a each including the CT gantry 20 and the PET gantry 10, as an example. However, the PET gantry 10 may be combined with another apparatus, other than the CT gantry 20. For example, the PET gantry 10 may be combined with a magnetic resonance imaging (MRI) apparatus. The MRI apparatus allows bloodstream evaluation. Thus, the MRI apparatus can detect a site or sites of the subject P in which the blood is stagnant. Such a blood stagnant site may be added to the determination condition for additional imaging. In this regard, the additional determination function 449 may determine whether additional imaging is to be performed according to a result of the determination on the sites included in the PET image based on the determination-criterion information and bloodstream information representing the subject P's bloodstream.

Further, the display control function 451 may display the blood stagnant site on the display 42 in a highlighted manner. For example, the display control function 451 may display the blood stagnant site in a different color.

### Second Modification

According to the first embodiment and the second embodiment, the additional determination function 449 determines whether additional imaging is to be performed based on the determination-criterion information and the PET image of each site extracted by the site extracting function 448. However, the additional determination function 449 may use additional information to determine whether additional imaging is to be performed. For example, the additional determination function 449 may additionally use patient information about the subject P being a patient to make a determination as to additional imaging. The patient information refers to information such as a clinical history, previous examination results, a body fat percentage, and an examination purpose.

As an example, the additional determination function 449 determines whether additional imaging is to be performed on the basis of a result of the determination on the PET image of each site extracted by the site extracting function 448 based on the determination-criterion information, and the patient information. The patient information is one example of subject information. For example, to examine a treated area for follow-up, the additional determination function 449 determines the sites other than the treated area as a low priority. For cancer metastasis examination, on the other hand, the additional determination function 449 determines the sites other than the treated area as a high priority. In this manner, the additional determination function 449 can make a determination as to additional imaging in accordance with the characteristics and/or the state of a patient.

### Third Modification

According to the first embodiment and the second embodiment, the additional determination function 449 may determine whether additional imaging is to be performed with respect to CT image data and PET image data, based on the determination-criterion information and noise contained in a CT image. For example, the additional determination function 449 may make a determination as to additional imaging based on noise contained in a CT image, e.g., occurrence or non-occurrence of artifacts and body-motion information on the subject P. In this manner the additional determination function 449 can determine whether to allow the PET gantry 10 to perform additional imaging without allowing the CT gantry 20 to perform additional imaging.

As an example, the correction-data obtaining function 443 corrects the PET image based on the CT image. However, the CT image may contain artifacts. In such a case, the correction-data obtaining function 443 cannot properly enhance the quality of the PET image obtained from the CT image by additional imaging. In view of this, the additional determination function 449 additionally use the occurrence or non-occurrence of artifacts and/or the body-motion information on the subject P for making a determination as to additional imaging. Thereby, the additional determination function 449 can determine whether to cause the CT gantry 20 to perform additional imaging.

According to at least one of the embodiments and modifications, it is possible to alleviate the burden of additional imaging.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising
processing circuitry configured to:
obtain a morphological image representing a morphology of a subject and a positron emission tomography (PET) image of the subject;
detect a region containing each of sites of the subject included in the PET image with reference to the morphological image;
determine whether additional imaging is to be performed for at least one of the sites included in the PET image, based on determination-criterion information containing a determination criterion, the determination criterion being a criterion based on which performing or not performing additional imaging is determined, and being set for each of the sites; and
output information according to a result of the determination.

2. The medical information processing apparatus according to claim 1, wherein the processing circuitry is configured to:
generate an examination plan for the additional imaging of an imaging area containing the at least one of the sites determined to be subject to additional imaging, and
output the examination plan.

3. The medical information processing apparatus according to claim 1, wherein
the processing circuitry is configured to issue a notification of a need for the additional imaging.

4. The medical information processing apparatus according to claim 3, wherein the processing circuitry is configured to:
receive an instruction for performing the additional imaging, and
output information according to the result of the determination, in response to receipt of the instruction.

5. The medical information processing apparatus according to claim 1, wherein
the processing circuitry is configured to determine a degree of priority of additional imaging with respect to the at least one of the sites determined to be subject to the additional imaging.

6. The medical information processing apparatus according to claim 5, wherein
the processing circuitry is configured to determine whether additional imaging is to be performed for the at least one of the sites based on:
the degree of priority,
a length of time defined by a medical agent administered to the subject, and
a length of time ensured for capturing the PET image of the subject.

7. The medical information processing apparatus according to claim 1, wherein
the processing circuitry is configured to determine whether additional imaging is to be performed based on:
the result of the determination as to the at least one of the sites included in the PET image based on the determination-criterion information, and
bloodstream information representing a bloodstream of the subject.

8. The medical information processing apparatus according to claim 1, wherein
the processing circuitry is configured to determine whether additional imaging is to be performed based on:
the result of the determination as to the at least one of the sites included in the PET image based on the determination-criterion information, and
subject information as to the subject.

9. The medical information processing apparatus according to claim 1, wherein
the processing circuitry is configured to determine whether additional imaging is to be performed with respect to the morphological image and the PET image, based on the determination-criterion information and noise contained in the morphological image.

10. The medical information processing apparatus according to claim 1, wherein
the processing circuitry is configured to display the obtained PET image and the PET image resulting from the additional imaging in a comparable manner.

11. A positron emission tomography apparatus comprising
processing circuitry configured to:
obtain a morphological image representing a morphology of a subject and a positron emission tomography (PET) image of the subject;
detect a region containing each of sites of the subject included in the PET image with reference to the morphological image;
determine whether additional imaging is to be performed for at least one of the sites included in the PET image, based on determination-criterion information containing a determination criterion, the determination criterion being a criterion based on which performing or not performing additional imaging is determined, and being set for each of the sites; and
perform the additional imaging according to a result of the determination.

12. A medical information processing method comprising:
obtaining a morphological image representing a morphology of a subject and a positron emission tomography (PET) image of the subject;
detecting a region containing each of sites of the subject included in the PET image with reference to the morphological image;
determining whether additional imaging is to be performed for at least one of the sites included in the PET image, based on determination-criterion information containing a determination criterion, the determination criterion being a criterion based on which performing or not performing additional imaging is determined, and being set for each of the sites; and
outputting information according to a result of the determination.
